(19) United States
(12) Patent Application Publication
(10) Pub. No.: US 2005/0177012 A1
(43) Pub. Date: Aug. 11, 2005

(54) Halocarbon production processes, halocarbon separation processes, and halocarbon separation systems

(75) Inventors: Mitchel Cohn, West Lafayette, IN (US); John Chien, West Lafayette, IN (US)

Correspondence Address: WELLS ST. JOHN P.S., 601 W. FIRST AVENUE, SUITE 1300, SPOKANE, WA, 99201, US

(73) Assignee: PCBU Services, Inc.

(21) Appl. No.: 11/038,982

(22) Filed: Jan. 19, 2005

Related U.S. Application Data
(63) Continuation-in-part of application No. 10/133,551, filed on Apr. 26, 2002.
Application No. 10/133,551 is a continuation of application No. 09/909,695, filed on Jul. 20, 2001, now abandoned.

(51) Int. Cl. C07C 19/08

(52) U.S. Cl. 570/237

(57) ABSTRACT

Halocarbon production processes are provided that can include reacting at least one C-2 halocarbon with at least one C-1 halocarbon in the presence of a phosphate to produce at least one C-3 chlorocarbon. The processes can include reacting ethylene with carbon tetrachloride in the presence of a phosphate. Halocarbon separation processes are provided that can include providing a reaction product that includes at least one saturated fluorocarbon and at least one unsaturated fluorocarbon and adding at least one hydrohalogen to produce a distillation mixture. Methods and materials are provided for the production and purification of halogenated compounds and intermediates in the production of 1,1,1,3,3-pentafluoropropane.

### CROSS REFERENCE TO RELATED APPLICATION

This application is a Continuation-in-Part of U.S. patent application Ser. No. 10/133,551, which was filed on Apr. 26, 2002 which was a continuation of U.S. application Ser. No. 09/909,695 filed Sep. 20, 2001; both of which are incorporated by reference herein.

### TECHNICAL FIELD

The present invention relates to methods and apparatus for the preparation and purification of halogenated hydrocarbons.

### BACKGROUND OF THE INVENTION

Numerous methods are known for the preparation of fluorocarbons. These methods vary widely, due in part to the different starting materials and reaction conditions involved.

For example, HFC-245fa is a known fluorocarbon that has found use as a foam blowing agent and also as a refrigerant. HFC-245fa has been prepared via the treatment of 1-chloro-3,3,3-trifluoropropene (CHCl═CHCF₃, HCFC-1233zd) with excess HF. However, purification of HFC-245fa from the resulting reaction mixture is difficult because HFC-245fa, HCFC-1233zd, and HF are difficult to separate by distillation.

U.S. Pat. No. 6,018,084 to Nakada et al., discloses a process in which 1,1,1,3,3-pentachloropropane (CCl₃CH₂CHCl₂) is reacted with HF in the gaseous phase, in the presence of a fluorination catalyst, to form HCFC-1233zd which is then reacted with HF in the gaseous phase to produce (HFC-245fa).

U.S. Pat. No. 5,895,825 to Elsheikh et al. discloses a process in which HCFC-1233zd is reacted with HF to form 1,3,3,3-tetrafluoropropene (CF₃CH═CHF) followed by further HF addition to form HFC-245fa.

Although the above described methods serve to produce HFC-245fa, these preparations, like the preparations of other fluorocarbons, are characterized by numerous disadvantages including expensive raw materials, poor yields, and poor selectivity which, render them difficult to use on a commercial scale.

### SUMMARY OF THE INVENTION

In brief, the present invention provides novel methods and materials for the preparation of halogenated hydrocarbons from readily available starting materials such as carbon tetrachloride and vinyl chloride. Processes for preparing precursors and intermediates in the production of HFC-245fa are described.

One aspect of the present invention is to provide a method for the production of HFC-245fa from readily available starting materials. In one embodiment of the present invention, 1,1,1,3,3-pentachloropropane is produced by supplying a reactor with a combination of carbon tetrachloride, vinyl chloride, and a metal chelating agent.

The 1,1,1,3,3-pentachloropropane is then dehydrochlorinated with a Lewis acid catalyst to produce 1,1,3,3-tetrachloropropene, which is then hydrofluorinated in multiple steps to produce HFC-245fa.

Halocarbon production processes are provided that can include reacting at least one C-2 halocarbon with a C-1 halocarbon in the presence of a phosphorous-comprising compound to produce a C-3 halocarbon. Embodiments of this process include reacting vinylidene chloride with carbon tetrachloride. Other processes can include reacting ethylene with carbon tetrachloride.

Halocarbon separation processes are provided that can include providing a mixture that includes a saturated fluorocarbon and an unsaturated fluorocarbon, and adding a hydrohalogen to this mixture to produce another mixture. The process can also include distilling the other mixture to separate at least a portion of the saturated fluorocarbon from the unsaturated fluorocarbon.

Halocarbon production systems are provided that can include a liquid phase reactor coupled to a first halocarbon reagent reservoir, with both a second halocarbon reagent reservoir and a phosphate reagent reservoir being coupled to the liquid phase reactor. The reactor can be coupled to an apparatus containing catalyst, with the reactor and reagent reservoirs being configured to provide reagent to the reactor and circulate reagent from the reactor through the apparatus and return the reagent to the reactor. Other systems can include a halocarbon product receiving reservoir coupled to a distillation apparatus, with a hydrohalogen reservoir coupled to the halocarbon product receiving reservoir.

### DESCRIPTION OF FIGURES

FIG. 1 is a diagram of a system according to an embodiment.

FIG. 2 is a diagram of a system according to an embodiment

### DETAILED DESCRIPTION OF THE PREFERRED EMBODIMENTS

This disclosure of the invention is submitted in furtherance of the constitutional purposes of the U.S. Patent Laws “to promote the progress of science and useful arts” (Article 1, Section 8).

According to an embodiment, a halocarbon production process is provided for preparing at least one C-3 halocarbon such as halogenated alkanes, by reacting a haloalkane and a haloalkene in the presence of a metal chelating agent. The haloalkane can be at least one C-1 halocarbon such as CCl₄, the haloalkene can be at least one C-2 halocarbon such as vinyl chloride, vinylidene chloride, and/or ethylene, and the metal chelating agent can be a phosphorous-comprising material. It was determined that other chelating agents containing phosphorous could be used. The phosphorous-comprising material can include a phosphorous-comprising compound such as tributyl phosphate. The halocarbon production process may be conducted in the presence of an iron-comprising material, such as elemental iron and/or iron wire. The ratio of haloalkane to haloalkene can be about 1.07:1. In an exemplary embodiment the C-2 halocarbon can include vinylidene chloride, the C-1 halocarbon can include carbon tetrachloride, and the molar ratio of the carbon tetrachloride to the vinylidene chloride can be between about 1.0 and 3.0. This reaction can occur at a temperature of about 105° C. and a reaction pressure of from 135-205 kPa. According to exemplary embodiments, the reaction pressure can be from about 230 kPa to about 253 kPa and reactants within the reactor can have a temperature of from about 95° C. to about 100° C. The reaction can produce 1,1,1,3,3-pentachloropropane. This compound can then be used to form HFC-245fa. One embodiment of the present reaction is demonstrated by the following non-limiting example.

### EXAMPLE 1

### Preparation of 1,1,1,3,3-Pentachloropropane

A 1 inch I.D. by 24 inch long continuous reactor was equipped with a sight glass, circulation pump, and pressure control valve. To the reactor was added 193 grams of iron wire, followed by the addition of carbon tetrachloride containing 3% by weight tributyl phosphate. The carbon tetrachloride was added to the reactor in an amount sufficient to fill the reactor to 60% of its total volume. The reactor was then heated to 105° C. and vinyl chloride was fed into the reactor until the 1,1,1,3,3-pentachloropropane concentration in the circulating product stream reached a concentration of 66% by weight. A mixture of 3% tributyl phosphate/carbon tetrachloride and vinyl chloride was then continuously fed into the reactor in a mole ratio of 1.07:1. Reaction pressure was controlled at 135-205 kPa and the product was removed by liquid level control. Analysis of the crude product indicated a 75% conversion to 1,1,1,3,3-pentachloropropane.

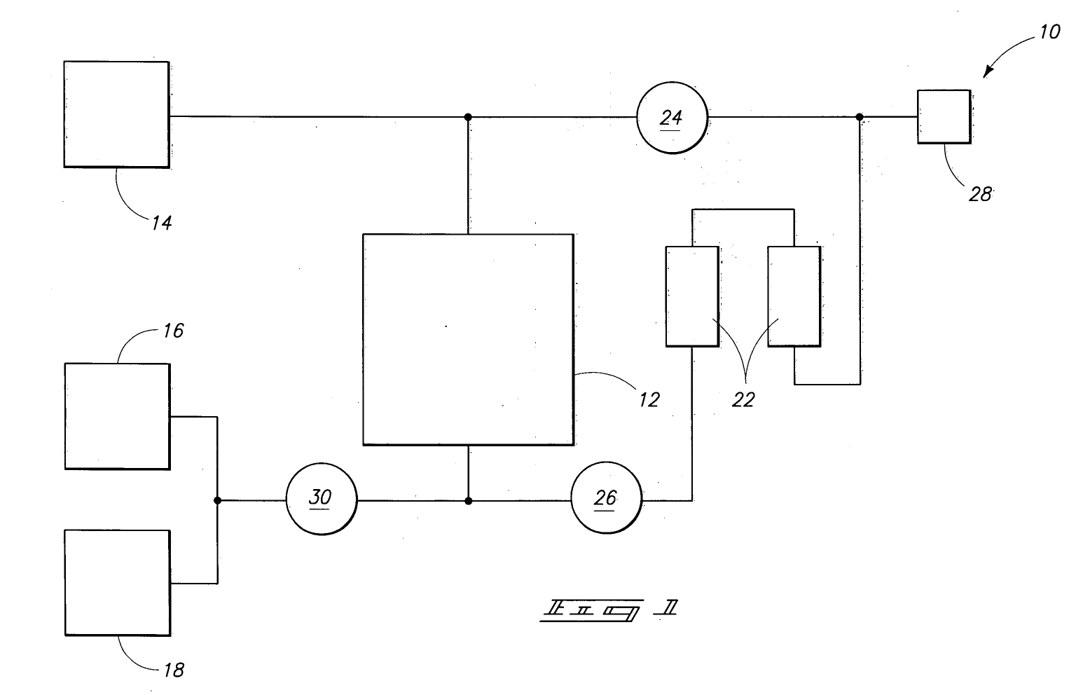

An embodiment of the present invention includes halocarbon production processes that can include reacting vinylidene chloride with carbon tetrachloride in the presence of a phosphorous-comprising material to produce at least one C-3 chlorocarbon. An exemplary embodiment of the halocarbon production processes is described with reference to FIG. 1. As depicted in FIG. 1, a halocarbon production system 10 includes a reactor 12 coupled to a first halocarbon reagent reservoir 14. Halocarbon reservoir 14 can be configured to store halocarbons such as the at least one C-2 halocarbon, including haloalkenes. In exemplary embodiments, reservoir 14 can contain haloalkenes such as ethylene, vinylidene chloride, and/or vinyl chloride. The halocarbon of reservoir 14 can be continuously added to reactor 12, in one embodiment.

Reactor 12 can be configured as a liquid phase reactor and, as such, reactor 12 can be manufactured of carbon steel and/or lined with PTFE (polytetrafluoroethylene), in one embodiment. Reactor 12 can also be lined with and/or constructed of stainless steel. Reactor 12 can be configured to receive reactants and convey products.

System 10 can also include another halocarbon reagent reservoir 16 coupled to a phosphate reagent reservoir 18, in one embodiment. Halocarbon reagent reservoir 16 and phosphate reagent reservoir 18 can be coupled to reactor 12. As exemplarily depicted in FIG. 1 reservoirs 16 and 18 can be coupled to reactor 12 at a point where products are conveyed from reactor 12. Reagent reservoir 18 can be configured to store the phosphorous-comprising material, such as tributyl phosphate. Reservoir 16 can be configured to store halocarbons and/or the at least one C-1 halocarbon such as haloalkanes including carbon tetrachloride. The reservoirs can be charged with nitrogen to facilitate the transfer of their contents to reactor 12. At least a portion of either of the halocarbons can be in the liquid phase during the reacting in reactor 12, according to exemplary embodiments.

Reagents from reservoirs 16 and 18 can be combined to form a reagent mixture 30. Reagent mixture 30 can include a halocarbon and a phosphorous-comprising material. Reagent mixture 30 can be combined with products from reactor 12 to form a reactant mixture 26.

System 10 can also include an apparatus 22 coupled to reactor 12. Apparatus 22 can include catalyst tubes. Apparatus 22 can be configured to contain a catalyst such as iron. Apparatus 22 can also be configured to have reaction mixture 26 circulated therethrough and returned to reactor 12. ,In exemplary embodiments, reactor 12, and reservoirs 14, 16, and 18 can be configured, as shown, to provide reagent contained within these reservoirs to reactor 12, and circulate reaction mixture 26 from reactor 12 through apparatus 22, and return the reaction mixture to reactor 12. In an exemplary process the reaction mixture can cycle back and forth between reactor 12 and apparatus 22. For example, reactants of reservoir 14 can be provided to reactor 12, exit reactor 12, and combine with reagent mixture 30 to from reactant mixture 26. Mixture 26 can flow through apparatus 22 and a slip stream 24 can be returned to reactor 12. Slip stream 24 can be combined with reagent from reservoir 14 before being returned to reactor 12. The flow through apparatus 22 can be about 1.2 meters per second. Reaction mixture 26 can include vinylidene chloride, a phosphorous-comprising material, and carbon tetrachloride, for example. In exemplary embodiments, upon exposure of the reaction mixture to the iron-comprising material within apparatus 22, one of ferrous chloride and/or ferric chloride may be formed. Either or both of these chloride compounds may catalyze the halocarbon production process.

According to exemplary embodiments, reaction mixture 26 can be filtered prior to being circulated through apparatus 22. Reactor 12 has a total internal volume and the reaction mixture can comprise less than 90% of the total internal volume of reactor 12. In other embodiments, the reaction mixture can comprise between about 70% and about 90% of the total internal volume of reactor 12, and in still other embodiments, the reaction mixture can comprise less than about 80% or less than about 70% of the total internal volume of reactor 12.

As exemplarily depicted in FIG. 1, system 10 can provide for the recovery of halocarbon product in reservoir 28. The recovery of halocarbon product can be facilitated through the use of separation assemblies such as distillation assemblies, including condensers, coupled to reactor 12. In one exemplary embodiment halocarbon product can be the remainder of reaction mixture 26 after removal of slip stream 24. A portion of the product obtained from reactor 12 can be flash evaporated.

Reservoir 14 can contain vinylidene chloride and reservoir 16 can contain carbon tetrachloride, in accordance with exemplary embodiments. The mole ratio of carbon tetrachloride to vinylidene chloride can be between about 1.0 and 3.0 and, in exemplary embodiments, 2.7. According to exemplary embodiments, where reservoir 14 contains vinylidene chloride, and reservoir 16 contains carbon tetrachloride, product reservoir 28 can contain a C-3 chlorocarbon such as hexachloropropane.

In exemplary embodiments, reservoir 14 can contain ethylene. Where reservoir 14 contains ethylene, and reservoir 16 contains carbon tetrachloride, product reservoir 28 can contain tetrachloropropane. It has been determined that the pressure within reactor 12 can impact the efficiency of the reaction and, more particularly, the production of by-products. For example, the pressure within reactor 12 can be less than 791 kPa and/or greater than 170 kPa. In other embodiments, the pressure within reactor 12 can be less than 998 kPa and/or greater than 377 kPa, and/or between 446 kPa and 653 kPa.

The temperature of the mixture within reactor 12 can affect the production of by-product. In exemplary embodiments, the temperature of the mixture within reactor 12 can be less than 115° C. and/or greater than 80° C., and in other embodiments, the temperature of the mixture within the reactor can be between 80° C. and about 115° C. The temperature of the mixture within the reactor can also be greater than about 105° C. According to an embodiment, where reservoir 14 contains vinylidene chloride and reservoir 16 contains carbon tetrachloride, the temperature of the mixture within reactor 12 can be maintained at about 90° C.

### EXAMPLE 2

### Preparation of Hexachloropropane

TABLE 1

| | | Mole | Mole/ | | Vol |
|---|---|---|---|---|---|
| | MW | Ratio | min | g/min | ml/min |
| Reactants Compound | | | | | |
| Vinylidene Chloride | 96.94 | 1.0000 | 0.2479 | 21.20 | 17.38 |
| (VDC) | | | | | |
| Carbon | 153.82 | 2.1200 | 0.5256 | 80.74 | 50.78 |
| Tetrachloride | | | | | |
| (CCl₄) | | | | | |
| Tributyl Phosphate | 266.32 | 0.0173 | 0.0091 | 2.42 | 2.48 |
| Total | | | | 104.36 | 70.64 |
| Products Compound | | | | | |
| Hexachloropropane | 250.40 | 0.7700 | 0.1909 | 47.80 | 28.12 |
| XS VDC | 96.80 | 0.0100 | 0.0025 | 0.24 | 0.20 |
| XS CCl₄ | 153.60 | 1.2500 | 0.3099 | 47.60 | 29.94 |
| By-products | 347.71 | 0.1000 | 0.0248 | 8.58 | 5.05 |
| Total | | | | 104.2255 | |

The data of Table 1 above is acquired using the following general description. The exemplary reactor is constructed of 25.4 cm, schedule 40, 316 stainless steel pipe with 150# class flanges. The reactor interior height is 66 cm face to face, thereby having the maximum capacity of 33.4 liters. The heads to this reactor are constructed of 25.4 cm, 150# blind flanges that are drilled and have nozzles welded thereto as necessary to accommodate the piping and instrumentation of the exemplary system. Four nozzles are on the upper head and one nozzle is on the bottom head of the reactor. The reactor has a “strap on” jacket or panel coil affixed thereto. Thermally conductive paste (Thermon) is applied between the jacket and the reactor. There is no liner in this reactor. The reactor has a working capacity of 7 gallons. It is operated at 70% of capacity or 18.9 liters. The pump is run at 12.9 liters per minute to achieve a 1.2 meters per second linear flow rate through a catalyst bed having a 1.9 cm inner diameter. Vinylidene chloride is fed directly into the top of the reactor. Exemplary instrumentation includes a level transmitter (radar), pressure transmitter (Hastelloy® diaphragm) and temperature probe (K type thermocouple). A pressure relief valve initially is installed on the reactor at reliefs of 1135.5 kPa and/or 652.9 kPa.

Exemplary vinylidene chloride from the reactor is transferred from the bottom nozzle via 2.5 cm PTFE lined pipe to a 37.9 liters per minute magnetically coupled centrifugal stainless steel pump. The exemplary design includes a flex joint before the pump to isolate vibration and allow for alignment. The vinylidene chloride is then passed through multiple catalyst tubes. The tubes are packed with iron wire, the iron wire forming a catalyst bed within the tubes. The catalyst tubes are assumed to be empty when calculating packing volume. The relative catalyst packing ratio can vary based on catalyst usage. The percent wire packing for a 1.9 cm pipe is 80% (20% void space) when using 1.44 mm diameter wire. For a 15 cm pipe the percent packing is 90% (10% void space) for the same size wire. Regardless, the linear flow velocity for the empty catalyst bed is 1.2 meters per second. The pump has a by-pass loop available on it to allow for maintaining a constant flow rate through the exemplary catalyst bed. The available catalyst surface area per unit volume is equivalent. The catalyst apparatus is five 2.44 meter sections for a total of 12.2 meter of apparatus, or one 2.44 meter section. From the catalyst bed, the mixture flows through a #10 mesh stainless steel strainer to remove pieces of iron wire that may have detached from the bed. The pump stream is kept below 90° C. by cooling the reactor via the jacket and adding brine cooling tubing around the pump head.

From the tubes, the vinylidene chloride is then combined with a CCl₄ and tributyl phosphate feed stream to form a reaction mixture. The reaction mixture is transferred to a heat exchanger with 0.65 square meters of surface area. A side of the heat exchanger is constructed of Hastelloy® C276 alloy. This heat exchanger heats the reaction mixture to 90° C. From the heat exchanger the reaction mixture is transferred to the exemplary reactor and subsequently cycled through the tubes as described above.

A crude product stream is taken off continuously after the pump discharge. A level transmitter in the reactor controls the rate at which this stream is taken off. This stream is initially transferred to the flash evaporator or to a cylinder that serves as lag storage between the process and the evaporator. The process runs at a steady state based on the above parameters and the composition of the crude product stream is as indicated in Table 1 above.

Another aspect of the present invention provides processes of preparing a halogenated propene by reacting a halopropane in the presence of a Lewis acid catalyst. The halopropane can be 1,1,1,3,3-pentachloropropane, the Lewis acid catalyst can be FeCl₃ and the halogenated propene product can be 1,1,3,3-tetrachloropropene. Other Lewis acid catalysts are expected to exhibit similar performance. The reactants can be combined at a temperature of 70° C. The halopropane can be produced from a reaction involving a haloalkane and a haloalkene, preferably CCl₄ and vinyl chloride respectively. The process can further comprise reacting the halogenated alkene, either in a single or multiple steps, to form HFC-245fa.

The temperature of the reaction is generally one which is preferably high enough to provide a desired amount and rate of conversion of the halogenated propene, and preferably low enough to avoid deleterious effects such as the production of decomposition products and unwanted by-products. The reaction is preferably carried out at a temperature between 30° C. and about 200° C. A more preferred range for the reaction is from about 55° C. to about 100° C. It will be appreciated that the selected temperature for the reaction will depend in part on the contact time employed; in general, the desired temperature for the reaction varies inversely with the contact time for the reaction. The contact time will vary depending primarily upon the extent of conversion desired and the temperature of the reaction. The appropriate contact time will, in general, be inversely related to the temperature of the reaction and directly related to the extent of conversion of halogenated propene.

The reaction can be conducted as a continuous flow of the reactants through a heated reaction vessel in which heating of the reactants may be effected. Under these circumstances the residence time of the reactants within the vessel is desirably between about 0.1 seconds and 100 hours, preferably between about 1 hour and about 20 hours, more preferably about 10 hours. The reactants may be preheated before combining, or may be mixed and heated together as they pass through the vessel. Alternatively, the reaction may be carried out in a batch process with contact time varying accordingly. The reaction can also be carried out in a multistage reactor wherein gradients in temperature, mole ratio, or gradients in both temperature and mole ratio are employed.

The weight percent of the Lewis acid catalyst can be determined by practical considerations. A preferred range for the weight percent of catalyst is: from 0.01% to 40% by weight, based on the weight of halogenated propene and Lewis acid catalyst mixture; preferably about 0.05% to about 1%, with a weight percent of from about 0.05% to about 0.5% by weight; particularly about 0.1% by weight being most preferred. Suitable Lewis acid catalysts include any of the commonly known Lewis acids and include, for example, BCl₃, AlCl₃. TiCl₄, FeCl₃, BF₃, SnCl₄, ZnCl₂, SbCl₅, and mixtures of any two or more of these Lewis acids.

The reaction can be carried out at atmospheric pressure or at subatmospheric or superatmospheric pressures. The use of subatmospheric pressures can be especially advantageous in reducing the production of undesirable products. By way of non-limiting example, one embodiment of this reaction is demonstrated as follows.

### EXAMPLE 3

### Dehydrochlorination of 1,1,1,3,3-Pentachloropropane

Into a 500 ml round bottom flask was added 270 grams of 1,1,1,3,3-pentaclororopropane. To this was added 2.7 grams of anhydrous FeCl₃ to form a slurry. The slurry was stirred under a pad of nitrogen and heated to 70° C. The solution was sampled at 30 minute intervals to give 1,1,3,3-tetrachloropropene with the following conversions and selectivity:

| Time (min.) | Conversion (area %) | Selectivity (%) |
|---|---|---|
| 30 | 62.52 | 100 |
| 60 | 83.00 | 100 |
| 90 | 90.7 | 99.68 |
| 120 | 94.48 | 99.32 |

According to another embodiment, reactions of the present invention can be combined to perform a process for the production of HFC-245fa comprising the following steps: (1) reacting carbon tetrachloride with vinyl chloride to produce 1,1,1,3,3-pentachloropropane; (2) dehydrochlorinating the 1,1,1,3,3-pentachloropropane with a Lewis acid catalyst to produce 1,3,3,3-tetrachloropropene; (3) fluorinating the 1,3,3,3-tetrachloropropene to produce HCFC-1233zd; and (4) fluorinating the HFC-1233zd to produce HFC-245fa. The fluorination reaction of 1,3,3,3-tetrachloropropene with HF, step (3) of the process of the present invention, and the fluorination reaction of HCFC-1233zd with HF, step (4) of the process of the present invention have previously been described. (e.g., U.S. Pat. No. 5,616,819 to Boyce, et al.).

Other embodiments of the present invention address the difficulty of separating certain halogenated organic compounds and HF, such as HFC-245fa and HCFC-1233zd, for example. The normal boiling points of HFC-245fa and HCFC-1233zd are 15° C. and 20.8° C. respectively. It is expected that normal distillation would separate the HFC-245fa as the lights or overhead product and the HCFC-1233zd as the heavies or bottoms product. However this expected separation does not occur; HFC-245fa and HCFC-1233zd form an azeotropic and/or an azeotrope-like composition upon attempted separation by distillation.

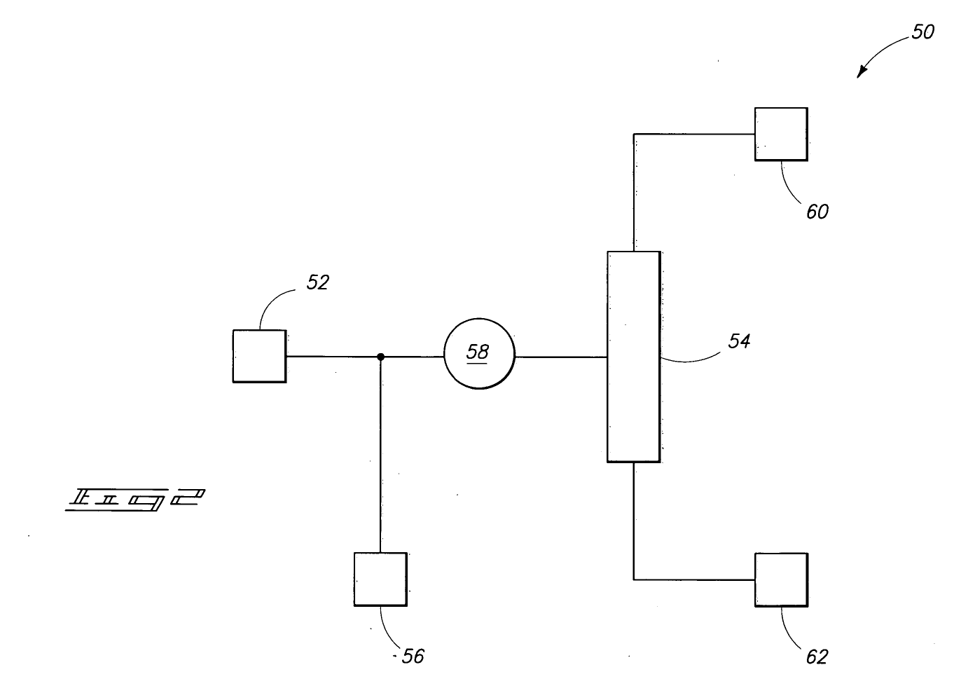

An exemplary embodiment of a halocarbon separation process is described with reference to FIG. 2. As depicted in FIG. 2, a halocarbon separation system 50 includes a distillation apparatus 54 coupled to a crude product reservoir 52 and a hydrohalogen reservoir 56. Apparatus 54 can be configured to separate components of mixtures based on the boiling points of the components within the mixtures. In exemplary embodiments, distillation apparatus 54 can include any apparatus that can be configured to have its temperature predetermined. Apparatus 54 can also be coupled to a product reservoir 62 and a by-product reservoir 60.

Reservoir 52 can contain a mixture comprising at least one saturated fluorocarbon and at least one unsaturated fluorocarbon. This mixture in certain embodiments can be produced by exposing at least one chlorocarbon to at least one halogenation exchange reagent in the presence of at least one catalyst. In specific embodiments the chlorocarbon can include CCl₃CH₂CCl₃, the halogenation exchange reagent can include HF and the catalyst can comprise Sb. It is generally accepted that the product of this reaction can result in a mixture including the saturated fluorocarbon such as CF₃CH₂CF₃ and the unsaturated fluorocarbon such as CF₃CH═CF₂. In certain exemplary embodiments, the unsaturated fluorocarbon can be a by-product produced during the production of the saturated fluorocarbon.

In exemplary embodiments the saturated and unsaturated fluorocarbons can form an azeotrope or azeotrope-like composition. As used herein, the term “azeotrope-like” is intended in its broad sense to include both compositions that are strictly azeotropic and compositions that behave like azeotropic mixtures. From fundamental principles, the thermodynamic state of a fluid is defined by pressure, temperature, liquid composition, and vapor composition. An azeotropic mixture is a system of two or more components in which the liquid composition and vapor composition are equal at the stated pressure and temperature. In practice, this means that the components of an azeotropic mixture are constant boiling and cannot be separated during a phase change.

Azeotrope-like compositions are constant boiling or essentially constant boiling. In other words, for azeotrope-like compositions, the composition of the vapor formed during boiling or evaporation is identical, or substantially identical, to the original liquid composition. Thus, with boiling or evaporation, the liquid composition changes, if at all, only to a minimal or negligible extent. This is to be contrasted with non-azeotrope-like compositions in which, during boiling or evaporation, the liquid composition changes to a substantial degree. All azeotrope-like compositions of the invention within the indicated ranges as well as certain compositions outside these ranges are azeotrope-like.

Reservoir 56 can contain at least one hydrohalogen. An exemplary hydrohalogen includes HF. Referring to an exemplary aspect, materials contained in reservoir 52 and 56 can be combined to produce a mixture comprising the saturated fluorocarbon, the unsaturated fluorocarbon and the hydrohalogen. This mixture can then be transferred to distillation apparatus 54 where it is separated. Within apparatus 54 this mixture can be distilled to separate at least a portion of the saturated fluorocarbon from the unsaturated fluorocarbon.

A product rich in unsaturated fluorocarbon can be collected at the upper portion of distillation apparatus 54 as primarily a gas and then subsequently condensed and stored in reservoir 60. In certain exemplary embodiments compounds collected within reservoir 60 can subsequently be transferred as a fluorocarbon mixture for a fluorocarbon production process and/or the HF can be separated from the compounds and used in the same or other processes.

A product rich in saturated fluorocarbon can be collected at the lower portion of distillation apparatus 54 and stored in reservoir 62. In certain exemplary embodiments reservoir 62 can contain primarily HF and saturated fluorocarbons. The product within reservoir 62 can include less than 2.4% unsaturated fluorocarbon or less than the azeotrope or azeotrope-like amount of unsaturated fluorocarbon, where the saturated and unsaturated fluorocarbons in specific quantities can form an azeotrope or azeotrope-like composition. With respect to the product in reservoir 62, this product can either be utilized as a final product containing primarily saturated fluorocarbons and/or processed subsequently by further purification methods.

Another process described provides methods for removing HF from a mixture containing HF and a halogenated hydrocarbon by combining the mixture with a solution of inorganic salt and HF and recovering a substantially pure halogenated hydrocarbon. In preferred embodiments of the process, the halogenated hydrocarbon is HFC-245fa and the inorganic salt is spray dried KF, the temperature of the solution of inorganic salt and HF is approximately 90° C., and the mole ratio of inorganic salt to HF is from about 1:2 to about 1:4. Other embodiments of the present invention include the utilization of halogenated hydrocarbons that are crude products of halogenation reactions, such as crude HFC-245fa, having impurities of HCFC-1233zd and HF. The present invention also provides an efficient method for regenerating the solution of inorganic salt and HF by removing HF until the mole ratio of inorganic salt to HF is about 1:2. The HF can be removed by flash evaporation.

Without being bound to any theory, it is contemplated that treating a mixture of HF and HFC-245fa with the HF/inorganic salt solution results in absorption of HF by the HF/inorganic salt solution that corresponds to a reduced amount of free HF present with HFC-245fa. Subsequent distillation of the HF/inorganic salt solution treated mixture of HF and HFC-245fa produces essentially pure HFC-245fa, and avoids the separation difficulties associated with mixtures of HF and HFC-245fa. Suitable inorganic salts include alkali metal fluorides such as sodium and potassium fluoride. Suitable molar ratios of alkali metal fluoride to HF range from 1:1 to 1:100, more preferably from 1:2 to 1:4.

The temperature of the HF/inorganic salt solution of this process is preferably between about 50° C. and about 150° C., and more preferably between about 75° C. and about 125° C. The process step can be conducted as a continuous flow of reactants through a heated reaction vessel in which heating of the reactants may be effected. The mixture containing the HF and HFC-245fa may be preheated before combining, or may be mixed and heated together with the HF/inorganic salt solution as they pass through the vessel. The substantially HF free halogenated hydrocarbon may be recovered as a gas or a liquid.

Following the absorption of HF, the resultant HF/inorganic salt solution can be treated to allow recovery of the absorbed HF and regeneration of the original HF/inorganic salt solution. Embodiments of the present invention are demonstrated below by way of non-limiting examples.

### EXAMPLE 4

### HF Removal from HFC-245fa/HF

To a 600 ml reactor was charged 200 grams of spray-dried KF and 147.47 grams of HF (1:2 mole ratio). The solution was held at 90° C. while 247.47 grams of a 1,1,1,3,3-pentafluoropropane/HF mixture (21.85 wt % HF) was allowed to bubble through the reactor. The analysis of material, such as vapor, exiting the reactor indicated that it was approximately 97% (w/w) HFC-245fa; the remainder of the material was primarily HF.

### EXAMPLE 5

### Regeneration of HF/KF Mixture (HF Recovery)

Following treatment of the HFC-245fa/HF mixture, the HF/KF solution was warmed to 170° C. and HF flashed into a water scrubber until the pressure dropped from 951 kPa to 101.3 kPa. Titration of the KF solution showed a KF/HF mole ratio of 1:2.06

### EXAMPLE 6

### Isolation of 1,1,1,3,3-Pentafluoropropane

A mixture of HFC-245fa and HF (20.26 wt %) was fed into a reactor with a 2.4 HF/KF (mole ratio) solution at 118° C. After absorbing HF only 1.94% HF remained in the HFC-245fa. The HF was recovered by vacuum evaporation of the ₓHF/KF solution (molar ratio) as per Example 5, preferably where x≧22, usually 2-3.

In another embodiment, the present invention provides processes for separating HFC-245fa from a mixture that includes HFC-245fa and HCFC-1233zd. The mixture of HFC-245fa and HCFC-1233zd can be the product of a halogenation reaction. In one embodiment, a mixture of HFC-245fa and HCFC-1233zd is distilled to produce a first distillate rich in HCFC-1233zd, and a bottom rich in HFC-245fa, and the bottom is distilled further to produce a second distillate of essentially HCFC-1233zd free HFC-245fa. In another embodiment, the first distillate is recycled to a halogenation reaction. This process is demonstrated by way of non-limiting example 7 below.

### EXAMPLE 7

### Azeotropic Distillation of HFC-245fa and HCFC-1233zd

A mixture containing primarily HFC-245fa to be purified by distillation of a lights and a heavies cut is fed to two distillation columns. The first distillation column removes the lights overhead, and the bottoms of the first distillation column is fed to a second distillation column. The purified HFC-245fa is removed as the product stream from the overhead of the second distillation column, and the heavies are removed from the bottom of the second distillation column. The concentration of HCFC-1233zd in the overhead stream of the first distillation column was analyzed as 98.36% HFC-245fa with 0.3467% HCFC-1233zd by weight, and this overhead stream can be incinerated or recycled to step (4) of the process (fluorination of 1-chloro-3,3,3-trifluoropropene). The bottoms of the first distillation column was 99.04% HFC-245fa and 43 ppm HCFC-1233zd, and the purified product (HFC-245fa) from the overhead stream of the second distillation column was 99.99% HFC-245fa and 45 ppm HCFC-1233zd.

In another embodiment the present invention provides processes for separating HFC-245fa from a mixture containing HFC-245fa and HCFC-1233zd. According to one embodiment the mixture is distilled in the presence of HF to produce a HFC-245fa bottom free of HCFC-1233zd and a distillate. In another embodiment the distillate is recycled to an HFC-245fa production reaction. The following non-limiting examples are demonstrative of this process.

### EXAMPLE 8

### Purification of Crude 1,1,1,3,3-Pentafluoropropane

A mixture of crude 1,1,1,3,3-pentafluoropropane containing a small amount of HF was fed into a 3.8 cm×305 cm long distillation column equipped with a condenser and a pressure control valve. The mixture was put into total reflux and then sampled. The results were as follows:

| | | HFC- | HCFC- | | HF wt | |
|---|---|---|---|---|---|---|
| | Light | 245fa | 1233zd | Heavies | % | Comments |
| Feed | ND | 99.83 | 0.0898 | 0.0803 | 3.66 | |
| Top gas | 0.0380 | 98.4143 | 1.4389 | 0.0942 | 3.47 | Not near |
| vapor | | | | | | azeotrope |
| Top Liquid | ND | 99.3024 | 0.6269 | 0.0707 | 19.55 | Not near |
| (reflux) | | | | | | azeotrope |
| Bottom | ND | 99.9405 | ND | 0.0595 | 2.3 | |
| liquid | | | | | | |

### EXAMPLE 9

### Purification of Crude 1,1,1,3,3-Pentafluoropropane

A similar test was performed as in Example 8. The results are shown below:

| | | HFC- | HCFC- | | | |
|---|---|---|---|---|---|---|
| | Light | 245fa | 1233zd | Heavies | HF wt % | Comments |
| Feed | ND | 99.45 | 0.0758 | 0.4211 | 3.83 | |
| Top gas | ND | 99.78 | 0.191 | 0.01 | 16.95 | Not near |
| vapor | | | | | | azeotrope |
| Top Liquid | ND | 99.81 | 0.164 | 0.025 | 21.21 | Not near |
| (reflux) | | | | | | azeotrope |
| Bottom | ND | 99.64 | 0.007 | 0.393 | 1.95 | |
| liquid | | | | | | |

In accordance with a preferred embodiment of the present invention, HFC-245fa is produced by: (1) reacting carbon tetrachloride (CCl₄) with vinyl chloride (CH₂═CHCl) to produce 1,1,1,3,3-pentachloropropane (CCl₃CH₂CHCl₂); (2) contacting the 1,1,1,3,3-pentachloropropane with a Lewis acid catalyst to produce 1,3,3,3-tetrachloropropene (CHCl═CHCCl₃); (3) fluorination of 1,3,3,3-tetrachloropropene with HF in the liquid phase to produce HCFC-1233zd (CF₃CH═CHCl); (4) fluorination of HCFC-1233zd with HF in the liquid phase in the presence of a fluorination catalyst to produce a mixture of HFC-245fa, HF and HCFC-1233zd; (5) treatment of the product mixture from step (4) with an HF/inorganic salt solution to produce a crude product mixture containing HFC-245fa as the major component and minor amounts of HF and HCFC-1233zd; (6) distilling the product mixture from step (5) to produce a bottoms product containing HFC-245fa and a distillate portion containing HF and HCFC-1233zd; and (7) final purification of the bottoms product from step (6) to remove traces of acid, water, or other by-products from the HFC-245fa product.

According to another embodiment the method of separating the product from by-products, step (6) of the process of the present invention, includes the separation and recovery of HFC-245fa from the product mixture resulting from step (5), such as by distillation of the mixture to produce bottoms containing the HFC-245fa, and a distillate by-product mixture containing HF and olefinic impurities. Batch or continuous distillation processes are suitable for these preparations.

Another embodiment of the present invention includes a further purification step (7), wherein the HFC-245fa, isolated as a bottoms product from step (6), is purified via water scrubbing and distillation to remove residual traces of moisture and/or acid. Numerous processes are well known in the art and can be employed for the removal of residual amounts of acid and water, for example treatment with molecular sieves and the like.

Step (7) can be accomplished by first scrubbing the bottoms product from step (6) and then separating the product by distillation. Scrubbing can be accomplished either by scrubbing the bottoms product with water and then, in a separate step, neutralizing the acid with caustic until the pH is neutral, e.g., 6-8, or by scrubbing in a single step with water and caustic.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

1. A halocarbon production process comprising a reaction of at least one C-2 halocarbon with at least one C-1 halocarbon in the presence of a phosphorous-comprising material to produce at least one C-3 halocarbon, wherein the at least one C-2 halocarbon comprises one or more of vinylidene chloride, ethylene, and vinyl chloride.

2. The process of claim 1 wherein:
the at least one C-2 halocarbon comprises vinylidene chloride;
the at least one C-1 halocarbon comprises carbon tetrachloride; and
a molar ratio of the carbon tetrachloride to the vinylidene chloride is between about 1.0 and 3.0.

3. The process of claim 1 wherein the phosphorous-comprising material comprises at least one phosphorous-comprising compound.

4. The process of claim 1 wherein the at least one phosphorous-comprising compound comprises tributyl phosphate.

5. The process of claim 1 wherein the reaction of the at least one C-2 halocarbon with the at least one C-1 halocarbon is conducted in the presence of an iron-comprising material.

6. The process of claim 5 wherein the reaction of the at least one C-2 halocarbon with the at least one C-1 halocarbon is conducted in the presence of both the iron-comprising material and the phosphorous-comprising material.

7. The process of claim 5 wherein the iron-comprising material comprises elemental iron.

8. The process of claim 5 wherein the iron-comprising material comprises iron wire.

9. The process of claim 1 wherein at least a portion of one or both of the at least one C-2 and C-1 halocarbons are in a liquid phase during the reaction.

10. The process of claim 1 wherein:
the at least one C-2 halocarbon comprises vinylidene chloride;
the at least one C-1 halocarbon comprises carbon tetrachloride; and
the at least one C-3 halocarbon comprises hexachloropropane.

11. The process of claim 1 wherein the reaction comprises:
providing a mixture comprising the at least one C-2 and C-1 halocarbons and the phosphorous-comprising material within a reactor; and
transferring the mixture from the reactor to a catalyst container.

12. The process of claim 11 wherein the transferring comprises circulating the mixture between the reactor and the catalyst container.

13. The process of claim 12 wherein the at least one C-2 halocarbon is continuously added to the reactor during the cycling.

14. The process of claim 11 wherein:
the at least one C-2 halocarbon comprises vinylidene chloride; and
the at least one C-1 halocarbon comprises carbon tetrachloride.

15. The process of claim 14 wherein the reactor comprises a total internal volume and the mixture comprises less than 90% of the total internal volume of the reactor.

16. The process of claim 14 wherein the reactor comprises a total internal volume and the mixture comprises between about 70% and about 90% of the total internal volume of the reactor.

17. The process of claim 14 wherein the reactor comprises a total internal volume and the mixture comprises less than about 80% of the total internal volume of the reactor.

18. The process of claim 14 wherein the reactor comprises a total internal volume and the mixture comprises greater than about 70% of the total internal volume of the reactor.

19. The process of claim 11 wherein:
the at least one C-2 halocarbon comprises ethylene; and
the at least one C-1 halocarbon comprises carbon tetrachloride.

20. The process of claim 19 wherein a pressure within the reactor is less than 1135.5 kPa.

21. The process of claim 19 wherein a pressure within the reactor is greater than 170.3 kPa.

22. The process of claim 19 wherein a pressure within the reactor is less than 997.6 kPa.

23. The process of claim 19 wherein a pressure within the reactor is greater than 377.1 kPa.

24. The process of claim 19 wherein a pressure within the reactor is less than 790.8 kPa.

25. The process of claim 19 wherein a pressure within the reactor is between about 446.1 kPa and about 652.9 kPa.

26. The process of claim 19 wherein a temperature of the mixture within the reactor is less than 115° C.

27. The process of claim 1 9 wherein a temperature of the mixture within the reactor is greater than 80° C.

28. The process of claim 19 wherein a temperature of the mixture within the reactor is between about 80° C. and about 115° C.

29. The process of claim 19 wherein a temperature of the mixture within the reactor is greater than about 105° C.

30. The process of claim 19 wherein the reactor comprises a total internal volume and the mixture comprises less than 90% of the total internal volume of the reactor.

31. The process of claim 19 wherein the reactor comprises a total internal volume and the mixture comprises between from about 50% to about 70% of the total internal volume of the reactor.

32. The process of claim 19 wherein the reactor comprises a total internal volume and the mixture comprises greater than about 20% of the total internal volume of the reactor.

33. A halocarbon production process comprising:
preparing a reaction mixture, the reaction mixture comprising:
at least one C-2 halocarbon, the at least one C-2 halocarbon comprising one or more of vinylidene chloride, ethylene, and vinyl chloride;
a phosphorous-comprising material; and
at least one C-1 halocarbon;
exposing the reaction mixture to an iron-comprising material; and
recovering at least one C-3 halocarbon.

34. The process of claim 33 wherein the preparing the reaction mixture comprises:
preparing a solution comprising the at least one C-1 halocarbon and the phosphorous-comprising material; and
combining the solution with the at least one C-2 halocarbon to produce the reaction mixture.

35. The process of claim 33 wherein:
the at least one C-2 halocarbon comprises vinylidene chloride;
the at least one C-1 halocarbon comprises carbon tetrachloride; and
the phosphorous-comprising material comprises at least one phosphorous-comprising compound.

36. The process of claim 35 wherein the at least one phosphorous-comprising compound comprises tributyl phosphate.

37. The process of claim 33 wherein:
the at least one C-2 halocarbon comprises ethylene;
the at least one C-1 halocarbon comprises carbon tetrachloride; and
the phosphorous-comprising material comprises at least one phosphorous-comprising compound.

38. The process of claim 37 wherein the at least one phosphorous-comprising compound comprises tributyl phosphate.

39. The process of claim 33 wherein:
the at least one C-2 halocarbon comprises vinyl chloride;
the at least one C-1 halocarbon comprises carbon tetrachloride; and
the phosphorous-comprising material comprises at least one phosphorous-comprising compound.

40. The process of claim 39 wherein the at least one phosphorous-comprising compound comprises tributyl phosphate.

41. The process of claim 33 wherein the at least one C-1 halocarbon comprises carbon tetrachloride and the phosphorous-comprising material comprises tributyl phosphate.

42. The process of claim 33 wherein the iron-comprising material comprises iron wire.

43. The process of claim 33 wherein, upon exposure of the reaction mixture to the iron-comprising material, one or both of ferrous chloride and ferric chloride are formed, either or both of the ferrous chloride and ferric chloride catalyzing the process.

44. A halocarbon separation process comprising:
providing a first mixture comprising at least one saturated fluorocarbon and at least one unsaturated fluorocarbon;
adding at least one hydrohalogen to the first mixture to produce a second mixture comprising the at least one saturated fluorocarbon, the at least one unsaturated fluorocarbon, and the at least one hydrohalogen; and
distilling the second mixture to separate at least a portion of the at least one saturated fluorocarbon from the at least one unsaturated fluorocarbon.

45. The process of claim 44 wherein the at least one unsaturated fluorocarbon comprises a by-product and/or feed produced during the production of the at least one saturated fluorocarbon.

46. The process of claim 44 wherein the first mixture is produced by exposing at least one chlorocarbon to at least one halogenation exchange reagent in the presence of at least one catalyst.

47. The process of claim 46 wherein the at least one chlorocarbon comprises CCl₃CH₂CCl₃, the at least one halogenation exchange reagent comprises HF and the at least one catalyst comprises Sb.

48. The process of claim 44 wherein the at least one saturated fluorocarbon comprises CF₃CH₂CF₃ and the at least one unsaturated fluorocarbon comprises CF₃CH═CF₂.

49. The process of claim 48 wherein the at least one hydrohalogen comprises HF.

50. The process of claim 44 wherein:
the at least one saturated and unsaturated fluorocarbons can form an azeotrope or azeotrope-like composition; and
the distilling further comprises recovering a third mixture comprising less than the azeotrope or azeotrope-like amounts of the at least one unsaturated fluorocarbon.

51. A halocarbon production system comprising:
a reactor coupled to first and second halocarbon reagent reservoirs;
a phosphorous reagent reservoir coupled to the reactor; and
a catalyst container coupled to the reactor, wherein the reactor and reagent reservoirs are configured to provide reagent to the reactor and circulating the reagent between the reactor and the catalyst container.

52. The system of claim 51 wherein the first halocarbon reagent reservoir is configured to contain vinylidene chloride.

53. The system of claim 51 wherein the reagent container is lined with polytetrafluoroethylene.

54. The system of claim 51 wherein the second halocarbon reagent reservoir is configured to contain carbon tetrachloride.

55. The system of claim 51 wherein the catalyst container is configured to contain an iron-comprising material.

56. The system of claim 55 wherein the iron-comprising material comprises elemental iron.

57. The system of claim 55 wherein the iron-comprising material comprises iron wire.
